(19) United States
(12) Patent Application Publication
(10) Pub. No.: US 2022/0111095 A1
(43) Pub. Date: Apr. 14, 2022

(54) ANTIMICROBIAL COMBINATIONS SYSTEM AND METHOD

(71) Applicant: Donald Griffith, Houston, TX (US)

(72) Inventor: Donald Griffith, Houston, TX (US)

(21) Appl. No.: 17/070,365

(22) Filed: Oct. 14, 2020

(51) Int. Cl. A61L 2/238; A01N 59/16

(52) U.S. Cl. CPC A61L 2/238; A61L 2300/404; A61L 2300/104; A01N 59/16

(57) ABSTRACT

An antimicrobial composition includes an organic extract, such as a witch hazel extract, and a colloid selected from metal, halogen, and combinations of these, such as a silver colloid. The witch hazel extract includes tannins in excess of about 10 mg/ml, such as hamamelitannin, and the colloid includes about 10,000 PPM of metal, such as silver. The antimicrobial composition may be applied to or instilled through an indwelling urinary catheter or other medical device.

### TECHNICAL FIELD

The invention generally relates to chemical combinations for antimicrobial action, and more particularly relates to organic extracts combined with silver ions or halogens for antimicrobial quorum signaling inhibition.

### BACKGROUND

Bacterial and other microbial infection is a major concern in medicine. These infections may present serious consequences, even life-threatening consequences in some instances, to patients. Various precautions and remedies exist, most notably cleansing and antibiotics. Effectiveness of these may be limited, however, in certain conditions. New antimicrobial compounds and uses would be advantageous.

Many patients can suffer from microbial infections. The aging and disabled (e.g., neurological, physical or cognitive) are particularly susceptible to infections, for example, due to urinary or other complications. Normal, periodic, volitional urinary bladder continence is often disrupted in the aging and disabled. Aging and childbirth can weaken the pelvic floor muscles in females causing incontinence issues. In males over the age of about 50, testosterone production slowly enlarges the prostate gland and slowly constricts and closes the urethral channel leading to urinary retention (i.e., inability to urinate). As can be understood, certain disabilities also may cause incontinence concerns.

Surgical procedures, such as transurethral resection of prostate gland, radical prostatectomy, pelvic floor reconstruction, use of artificial urinary sphincters, and others, may successfully rehabilitate bladder and urethral channel functions in some patients. Moreover, pads, pull-ups, and diapers (e.g., mostly with females, but also by males), or indwelling urinary catheters (e.g., mostly in males, who use suprapubic abdominal or urethral catheters), can be helpful for incontinence. These and other options, nevertheless, can lead to infection problems.

In patients with urinary catheters, catheter associated urinary tract infections (CAUTI) are particularly a problem. The National Institutes of Health (NIH), Center for Disease Control (CDC) and Food and Drug Administration (FDA) have all identified CAUTI as one of the most common and expensive infectious diseases in the United States. Prophylactic use of antibiotics to treat these infections has been discouraged, because of evolution of antibiotic-resistant microbes. Microbial colonization risks of indwelling catheters, however, remain a major source of concern. Those risks are similar regardless of whether the catheter is placed abdominally or transurethrally.

Causes of CAUTI may include microbial attachment of skin microbes to external surfaces of indwelling catheters, formation and growth of biofilm by attached microbes, daily migration of colony-rich biofilms principally on catheter external surfaces toward the bladder lumen, and colonization of bladder urine. Colonized urine frequently occurs in indwelling catheters, amounting to about 100% of chronic catheters have colonization after 20+ days of use. Consequently, long-term catheter users typically have colonized urine for as long as the catheter is indwelling.

Use of systemic antibiotics and antibiotic catheter coatings has been discouraged, for example, by FDA, NIH and CDC, because multiple trials have shown little continued sterility and frequent development of mutant microbial species that are resistant. It has become apparent that many of the microbial species that cause CAUTI owe much of their pathogenicity to quorum signaling and biofilm formation. Quorum signaling is a process in which bacteria communicate by secreting and sensing diffusible signaling molecules called “autoinducers.” When these signaling molecules exceed a threshold concentration level, they auto-stimulate genes that enable them to behave as a multicellular population, to benefit their survival. In staphylococci, as example, autoinducers upregulate the expression of multiple toxins and enhance the formation of biofilms. These phenotypes are the hallmark of staph pathogenesis and the cause of resistant persistent infections and even death.

Consequently, a significant and serious need exists for suitable new antimicrobial compounds and uses thereof. It would, therefore, be a significant improvement in the art and technology to provide new antimicrobial compounds and uses thereof. It would further be an improvement to provide new antimicrobial compounds for application to and use with urinary catheters, as well as other devices. It would also be a further improvement to provide new antimicrobial compounds and uses that do not significantly lead to resistant microbes and overuse of conventional antibiotics and similar conventional antimicrobial options.

### SUMMARY

An embodiment of the invention is an antimicrobial composition comprised of an organic extract, for non-exclusive example, witch hazel extract, and a colloid metal, for non-exclusive example, silver colloid, or halogen, or combination of these.

Another embodiment of the invention is a method of inhibiting bacterial attachment to a device including instilling at least about twice daily a formulation of about 1 to about 10 ml of an about 1:1 to at least about 1:2 dilution of an organic botanical extract combined with a liquid colloid selected from the group consisting of: metal, halogen, and combinations of these.

Yet another embodiment of the invention is a method of inhibiting bacteria attachment and replication on a medical device including applying a formulation of liquid witch hazel extract and liquid silver colloid to a surface of the medical device.

Another embodiment of the invention is a method of making a composition for inhibiting bacteria attachment to a device surface, preventing microbial formation of biofilm, inhibiting microbial replication, and enhancing microbial killing attribute. The method includes producing a witch hazel extract and combining the witch hazel extract with a silver colloid.

### DETAILED DESCRIPTION

Embodiments include organic extracts, particularly, witch hazel extracts, comprising tannin and/or gallic acid, combined with halogen and/or metal, in an ointment, liquid or other formulation. The novel combination has been determined to effectively inhibit quorum signaling of bacteria and act to inhibit microbial attachment and replication on cellular surfaces and on metallic and polymeric device surfaces. Through benchtop experimentation, the combination has been shown to be more effective as an antibacterial than the individual components. The formulations may take a variety of forms, including liquids, sprays, ointments, creams, colloids, and others.

### Witch Hazel Extract

Witch hazel (Hamamelis) is a genus of flowering plants in the family Hamamelidaceae. Four species are found in North America (H. ovalis, H. virginiana and H. vernalis), and one species each is found in Japan (H. japonica) and China (H. mollis). The leaves and bark of the North American witch hazel, Hamamelis virginiana, may be used to produce an astringent decoction. This witch hazel decoction can be an extract in liquid form. This concentrated extract liquid has been FDA approved, for example, for topical application to the nose, anus and skin.

Witch hazel liquid extract/concentrate can include such components as calcium oxalate, gallotannins, and safrole, and chemicals found in the essential oil (carvacrol, eugenol). Witch hazel for use as a topical can be a liquid, semisolid ointment, cream, gel or salve, as example. Witch hazel extracts and concentrates are widely sold by pharmacies, grocery stores, and others, and they serve many uses to ease discomfort and provide soothing.

A particular witch hazel extract in the embodiments, determined to provide beneficial attributes, is marketed over the counter as a product named whISOBAX™ available from Staph-Off Biotech, Inc. at staphoff.com, as example. The whISOBAX™ product comprises about 12.66 mg of gallic acid equivalent/ml. The planktonic minimum bactericidal concentration/minimum inhibitory concentration (MBC/MIC) for whISOBAX™ is about 0.31/0.15 mg/ml gallic acid equivalence method (GAE) and the minimum inhibitory concentration (MIC) for biofilm trapped bacteria is about 0.47 mg/ml GAE [˜3× higher than the planktonic level].

Determined to be a particular anti-microbial component of the witch hazel extracts is the hamamelitannin molecule. The hamamelitannin molecule is substantially as follows:

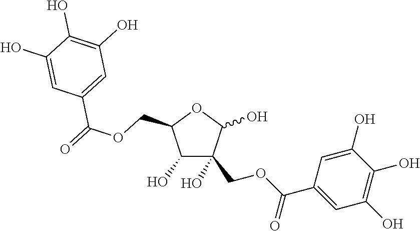 [SMILES: O=C(OC[C@H]1OC(O)[C@@](O)(COC(=O)c2cc(O)c(O)c(O)c2)[C@@H]1O)c1cc(O)c(O)c(O)c1]

Various analogues of hamamelitannin are possible and all are contemplated as suitable for the combinations in embodiments. Hamamelitannin acts as a quorum signaling inhibitor (QSI) that suppresses microbial biofilm formation and toxin production of bacteria.

Other anti-microbial active components of witch hazel include gallic acid and other phenolic compounds. Gallic acid, for example, may prevent bacterial growth by binding/disrupting cell membranes. The various components of witch hazel extract are effective in multiple gram negative and positive bacteria that commonly colonize indwelling catheters and the like. The primary function of witch hazel extract where deposited and in antimicrobial actions is to block microbial attachment to devices or biological surfaces, block biofilm formation, block toxins from being produce, and lower the MIC of other anti-microbials.

### Metals and Halogens

Silver ions, as well as other metals, for nonexclusive example, copper, tin, zinc and others, can have biocidal effects. Further, various halogens, such as, for nonexclusive example, iodine, chlorine and others, may contribute to anti-bacterial action. The mechanism of action of metals and halogens is believed to involve penetration into bacterial cells, turning deoxyribonucleic acid (DNA) molecules into a condensed form and thereby hindering cell replication.

Silver colloid, for example, has been approved by FDA for topical and oral use. Silver colloids can be a preferred drinking water sterilant when used in 20-100 mcg/liter concentrations. Russian space craft employ silver colloids to recycle urine and other body fluids. Other metals and colloids of metals are therefore effective for sterilization as well as other antimicrobial effects.

### Combination

The combination of witch hazel extract, with silver or other metal ion or colloid or halogen, has been determined in bench top testing to surprisingly serve as a more powerful antimicrobial than either agent alone. Testing in multiple dilutions, ranging from about 1:5, 1:10, 1:20, 1:40, 1:80 and others, indicates the efficacy of the combination against both gram+ and gram− microbial species.

A particular witch hazel extract, whISOBAX™, has about the following characteristics:

| Test | Specifications | | Method | Results |
|---|---|---|---|---|
| Identity | Hamamelis virginiana L. | | Macroscopy, microscopy and | Pass |
| | bark extract | | chromatography | |
| Ethanol/Water | 50% 190 proof | | Measurement by volume | Pass |
| ratio | EthOH/50% H2O | | | |
| Density @ 20° | 0.93-0.96 | | Measurement by weight/volume | 0.948 |
| Odor | Characteristic witch hazel, | | Examine organoleptic | Pass |
| | mildly aromatic, free from | | | |
| | Acetous Odor | | | |
| Color | Red/brown | | Examine (visual inspection) | Pass |
| Clarity | Transparent | | Examine (visual inspection) | Pass |
| pH @ 25° C. | 4.0-6.0 | | Measurement | 5.35 |
| Specific gravity | 0.902-0.99 | | Measurement | 0.949 |
| @ 20° C. | | | | |
| Tannins | >10 | mg/ml | HPLC | 17.3 mg/ml |
| Total Yeast and | <1000 | CFU/ml | USP <2021>, USP <2022> | Pass |
| Mold | | | Modified | |
| Aerobic Plate | <10,000 | CFU/ml | USP <2021>, USP <2022> | Pass |
| Count | | | Modified | |
| Total Coliforms | <10 | cfu/ml | USP <2021>, USP <2022> | Pass |
| | | | Modified | |

| | | | |
|---|---|---|---|
| Salmonella/E. | Negative | USP <2021>, USP <2022> | Pass |
| coli/S. aureus | | Modified | |
| Contaminants | <1.667/0.684/0.0834/0.334 | ICP-MS (EPA 200.8 [modified] | <0.002/0.001/0.002/0.005 |
| (Arsenic, | μg/ml | | μg/ml |
| Cadmium, Lead, | (3 ml daily dose) | | |
| Mercury) | | | |

This witch hazel extract, or similar variants of it, were employed in combinations in certain non-exclusive embodiments.

In tests, whISOBAX™ was combined with silver ion colloid. A particular silver ion colloid in tests, determined to provide beneficial attributes, is marketed over the counter as a product named Ultra Silver™ available from UltraSilver.com or Amazon.com, as example. Both whISOBAX™ and Ultra Silver™ are FDA certified as dietary supplements.

A particular silver colloid, UltraSilver™, has about the following characteristics:

| | |
|---|---|
| Pharmaceutical grade silver | 10,000 PPM (1000 μg/l) |
| Steam distilled water | </=~1 \| |
| Protein | Balance |

This silver colloid, or similar variants of it or of other metal or of halogen colloids, were employed in combinations in certain non-exclusive embodiments.

### Examples

Various dilutions of witch hazel extract to silver colloid have been effective in bench tests as broad range antimicrobial. The following table presents certain results obtained in lab testing:

URO-LITHIASIS LABORATORY, INC.

| | | | | whISOBAX ™ + Silver Colloid |
|---|---|---|---|---|
| | Dilutions | whISOBAX ™ | Silver Colloid | 10,000 PPM Combined |
| Microbial Species | | | | |
| Staphylococcus aureus | 1:10 | Susceptible | Susceptible | Susceptible |
| 155554A ATCC 27661 | 1:20 | Resistant | Intermediate | Susceptible |
| | 1:40 | Resistant | Resistant | Susceptible |
| | 1:80 | Resistant | Resistant | Susceptible |
| Staphylococcus epidermidis | 1:10 | Susceptible | Susceptible | Susceptible |
| 155556A ATCC 14990 | 1:20 | Resistant | Intermediate | Susceptible |
| | 1:40 | Resistant | Resistant | Susceptible |
| | 1:80 | Resistant | Resistant | Susceptible |
| Escherichia coli K-12 | 1:10 | Resistant | Susceptible | Intermediate |
| 124500 ATCC 10798 | 1:20 | Resistant | Susceptible | Intermediate |
| | 1:40 | Resistant | Intermediate | Intermediate |
| | 1:80 | Resistant | Resistant | Intermediate |
| Klebsiella pneumoniae | 1:10 | Susceptible | Susceptible | Susceptible |
| 155095A ATCC 132 | 1:20 | Intermediate | Susceptible | Susceptible |
| | 1:40 | Resistant | Susceptible | Susceptible |
| | 1:80 | Resistant | Susceptible | Susceptible |
| Pseudomonas aeruginosa | 1:10 | Susceptible | Susceptible | Susceptible |
| 155250A ATCC 27853 | 1:20 | Intermediate | Susceptible | Susceptible |
| | 1:40 | Resistant | Susceptible | Susceptible |
| | 1:80 | Resistant | Susceptible | Susceptible |
| Enterococcus faecalis | 1:10 | Susceptible | Susceptible | Susceptible |
| 155600A ATCC 19433 | 1:20 | Susceptible | Susceptible | Susceptible |
| | 1:40 | Susceptible | Intermediate | Susceptible |
| | 1:80 | Susceptible | Intermediate | Susceptible |
| Proteus mirabilis | 1:10 | Susceptible | Susceptible | Susceptible |
| 155239A ATCC 25933 | 1:20 | Intermediate | Susceptible | Susceptible |
| | 1:40 | Resistant | Susceptible | Susceptible |
| | 1:80 | Resistant | Susceptible | Susceptible |
| Proteus vulgaris | 1:10 | Susceptible | Susceptible | Susceptible |
| 155240A ATCC 13315 | 1:20 | Susceptible | Intermediate | Susceptible |
| | 1:40 | Susceptible | Intermediate | Susceptible |
| | 1:80 | Susceptible | Intermediate | Susceptible |
| Sterility Testing | | | | |
| whISOBAX, Undiluted | None | | | No Growth |
| Colloidal Silver, 10,000 PPM, Pure | None | | | No Growth |
| Diluent Used: Normal Saline | 1:10 | | | No Growth |
| | 1:20 | | | No Growth |
| | 1:40 | | | No Growth |
| | 1:80 | | | No Growth |

Where:
Susceptible = Visibly impedes bacterial growth,
Resistant = Did not inhibit bacterial growth,
Intermediate = Slight to moderate inhibition, in the presence of the agents
The foregoing analysis was performed for each of whISOBAX ™, alone; Ultra Silver ™, alone; and the combination of whISOBAX ™ and Ultra Silver ™ in same formulation at various dilutions. No new reactant and/or by-product molecules resulting from the combination of two water-soluble agents witch hazel extract and silver colloid were detected.

In the foregoing results, it appears that a combined formulation of witch hazel extract and silver colloid provides unexpectedly better antimicrobial effects than the individual components. In particular, a witch hazel extract, comprised of hamamelitannin, gallic acid, and possibly other constituents, combined with a silver colloid or other metal or halogen colloid, may provide antimicrobial propensities that present new and non-obvious benefits.

Combinations of a witch hazel extract, including of hamamelitannin, gallic acid, and others, and a metal and/or halogen colloid may be employed for a wide variety of topical and other uses as antimicrobial. As non-exclusive example, the combinations may be particularly beneficial in use for urinary concerns. Microbial colonization risks of indwelling catheters are pervasive as CAUTI. In fact, on the order of 100% of chronic catheter users would have colonized urine after about 20 or so days of indwelling catheter use. Use of systemic antibiotics, such as for catheter coatings, in vivo injections or insertions, or otherwise, in such situations can be problematic and lead to resistant strains.

A significant cause of CAUTI stems from quorum signaling and biofilm formation of bacteria. Quorum signaling is a process in which bacteria communicate via secretin and sensing diffusible signaling molecules called “autoinducers.” When these autoinducers exceed a threshold concentration level, the bacteria auto-stimulate genes that enable them to behave as a multicellular population, to benefit their survival. In staphylococci, as non-exclusive example, autoinducers upregulate the expression of multiple toxins and enhance the formation of biofilms. These phenotypes are the hallmark of staph pathogenesis and the cause of resistant persistent infections, which may lead to disability and even death. Quorum signaling induces much of the pathogenicity created by commonly encountered gram(+) and gram(−) bacteria in CAUTI.

Witch hazel extracts, including, for example, components of hamamelitannin, gallic acid, and others, are effective against some bacterial infections depending on the microbial species and the concentration of microbes/unity volume. Gallic acid prevents bacterial growth by binding/disrupting cell membranes, and hamamelitannin acts as a quorum signaling inhibitor to suppress biofilm formation and toxin production. In effect, these extracts, as well as the individual components (hamamelitannin and gallic acid, among others), block microbial attachment to catheters and other devices and/or biologic surfaces, block biofilm formation, block toxins from being produced, and lower MIC of other antimicrobials.

Silver colloids, as well as other metals and halogens, also have biocidal effects on a broad spectrum of bacterial pathogens. These are effective biocides because metal and halogen ions penetrate into bacterial cells, turning DNA molecules of the cells into a condensed form and thereby hindering cell replication.

Combinations of the components hamamelitannin and/or gallic acid, such as in witch hazel extracts, and metal and/or halogen, such as in silver colloid, appear to be more efficacious in combating bacterial infection than would be expected from results of each component acting alone. In a possible protocol for administration of the combination for CAUTI inhibition, whISOBAX™ and Ultra Silver™, in combination, are introduced as follows:

Patients with indwelling catheters will receive at least twice daily [AM/PM] instillations of a combination of whISOBAX™ and Ultra Silver™ colloid into the urinary bladder for at least two days and possibly for the duration of use of the indwelling catheter (or other device, as applicable). Following each instillation, bladder drainage will be capped/clamped to tolerance. Unclamping and drainage of bladder urine into collection bags will be initiated by the patient or caregiver when symptoms dictate a need for urine drainage.

A urine specimen will be collected each morning from the patient's indwelling catheter. The indwelling and continuous draining catheter may be clamped for 10-15 minutes prior to urine collection if needed. After the urine specimen is collected for urine culture testing, the indwelling catheter will be irrigated with 20+ ml of sterile water or saline to remove any mucous, blood, blood clots or crystalline debris from the bladder lumen. This irrigant will be discarded. Promptly thereafter, 10 ml of whISOBAX™/Ultra Silver™ colloid combination additive will be instilled into the bladder lumen and the catheter will be clamped to tolerance to allow the additive to remain in the bladder lumen for multiple hours. This whISOBAX™/Ultra Silver™ colloid combination additive instilled into the bladder is to remain within the bladder until the patient has urge to urinate. The patient or caregiver will then unclamp/uncap drainage tube and allow the bladder to drain into a collection bag. There is no need for additive placement into the bladder lumen except for the early morning and bed-time time periods at this time. Increase or decrease in the number and timing of daily bladder instillations may be variable according to implementation. Each of the collected urine specimens will have a label for patient ID #, date, time of specimen collection and earlier time that additive was added to the bladder lumen. Additional urine specimen(s) may or may not be collected for infection testing if symptoms prompt such concerns. The specimens will be refrigerated in a research refrigerator at the local institution and then transported to a lab for microbiology testing. As further protocol detail, an initial screening visit will be held. Informed consent and demographic date will be obtained. All antibiotics will be stopped at least two days prior to urine collection for the pre-treatment urine culture and for duration of the protocol procedure.

A second screening visit will then be held at least two days after the initial screening visit. A urine culture will be performed at this second screening visit.

Thereafter, in days 1, 2, and 3, after beginning urine culture from the second screening,
a. A urine dipstick test will be performed every morning.
b. The urine collection bag will be emptied prior to each bladder instillation.
c. The whISOBAX™/Ultra Silver™ colloid combination additive shall contain 2 ml of whISOBAX™ concentrate plus 2 ml of Ultra Silver™ silver colloid concentrate [10 mg/liter], plus also 6 ml of sterile water. The 10 ml shall be injected into and through the indwelling Foley catheter into the bladder in the morning and in the evening. Following instillation, the catheter will be capped, clamped or sealed in order to keep the additive within the bladder until the bladder is uncomfortably full.
e. All patients will be encouraged to keep the catheter clamped to tolerance after instillation of above fluids.
f. 10+ cc of urine will be collected directly from the indwelling bladder catheter each morning per day immediately before installation of the morning or evening fluid of whISOBAX™/Ultra Silver™/water combination. Early in day 1 collection will be made of urine specimen collection #1, followed by installation #1, and a second urine specimen collection #2 followed by installation #2 shall be made at bedtime of day 1. Third through sixth installations of whISOBAX™/Ultra Silver™/water combination shall similarly be made on days 2 and 3 (morning and bedtime), and early morning and bedtime collection of urine specimens (collections #3-6) will be made. An additional urine specimen collection #7 will be made early morning on day 4.
   Daily side effects will be logged and discussed with the patient.

In non-exclusive alternatives, the combination of hamamelitannin and/or gallic acid compositions, such as witch hazel extract, with metal or halogen colloid, such as silver colloid, can be of many liquid or gel forms. Additionally, the combination can be impregnated or disposed in or on tape, cloth, or other surface. The combination can be placed on devices, such as a catheter or other medical device of polyethylene, silicone, composites, or other materials. Variations of viscosity and flow characteristics, as well as elution and retention properties, are possible in the embodiments. In certain non-exclusive alternatives, the combinations of the embodiments are introduced by instillation into bodily cavities, such as bladder lumen, other vessel or other body part, coating or deposition on body part, coating or deposition on a medical device, such as a urinary catheter or other device, topical application to the skin or bodily area, or others.

In other non-exclusive alternatives, organic extracts may include hamamelitannin, gallic acid and/or other constituents. The organic extracts have quorum signaling inhibitor properties. In others, non-organic, synthetic or other constituents are possible. For non-exclusive example, synthetic hamamelitannin and/or gallic acid, and/or various analogues thereof, may be added, substituted or otherwise incorporated or employed. Of course, a wide variety of other variations are possible.

In the foregoing, the invention has been described with reference to specific embodiments. One of ordinary skill in the art will appreciate, however, that various modifications, substitutions, deletions, and additions can be made without departing from the scope of the invention. Accordingly, the specification and figures are to be regarded in an illustrative rather than a restrictive sense, and all such modifications substitutions, deletions, and additions are intended to be included within the scope of the invention. Any benefits, advantages, or solutions to problems that may have been described above with regard to specific embodiments, as well as device(s), connection(s), step(s) and element(s) that may cause any benefit, advantage, or solution to occur or become more pronounced, are not to be construed as a critical, required, or essential feature or element.

What is claimed is:

1. An antimicrobial composition, comprising:
an organic extract; and
a colloid selected from the group consisting of: metal, halogen, and combinations of these.

2. The composition of claim 1, wherein the organic extract is witch hazel extract.

3. The composition of claim 1, wherein the colloid is silver colloid.

4. The composition of claim 3, wherein the colloid is silver colloid.

5. The composition of claim 4, wherein the witch hazel extract comprises hamamelitannin.

6. The composition of claim 5, wherein the witch hazel extract comprises gallic acid.

7. The composition of claim 6, wherein the witch hazel extract to silver colloid components are in a ratio of about one of the following: 1:10, 1:20, 1:40, and 1:80.

8. The composition of claim 3, wherein the silver colloid is concentrated with silver to about 10,000 parts per million.

9. The composition of claim 2, wherein the witch hazel extract comprises tannins of greater than 10 mg/ml.

10. The composition of claim 8, wherein the witch hazel extract comprises tannins of greater than 10 mg/ml.

11. A method of inhibiting bacterial attachment to a device, comprising:
instilling at least about twice daily a formulation of about 1 to about 10 ml of an about 1:1 to at least about 1:2 dilution of an organic botanical extract to a liquid colloid selected from the group consisting of: metal, halogen, and combinations of these.

12. The method of claim 11, wherein the organic extract is witch hazel extract.

13. The method of claim 11, wherein the colloid is silver colloid.

14. The method of claim 12, wherein the colloid is silver colloid.

15. The method of claim 12, wherein the witch hazel extract comprises tannins of greater than 10 mg/ml.

16. The method of claim 13, wherein the silver colloid is concentrated with silver to about 10,000 parts per million.

17. The method of claim 13, wherein the ratio of witch hazel extract to silver colloid is at least about 1:1 to at least about 1:20.

18. A method of inhibiting bacteria attachment and replication on a medical device, comprising:
applying a formulation of liquid witch hazel extract and liquid silver colloid to a surface of the medical device.

19. The method of claim 18, wherein the medical device is selected from the group consisting of: an indwelling urinary catheter, a temporary component, a permanent component, a temporary implanted device, a permanently implanted device, and combinations of these.

20. A method of making a composition for inhibiting bacteria attachment to a device surface, preventing microbial formation of a biofilm, inhibiting microbial replication, and enhancing microbial killing, comprising:
producing a witch hazel extract; and
combining the witch hazel extract with a silver colloid.

21. The method of claim 20, further comprising:
creating the silver colloid.
